(19) United States
(12) Patent Application Publication
(10) Pub. No.: US 2007/0110795 A1
(43) Pub. Date: May 17, 2007

(54) Use of chitosan materials

(75) Inventors: Birger Hahnemann, Zossen (DE); Andrea Pahmeier, Zossen (DE); Philipp Sperling, Berlin (DE); Thomas Lotzbeyer, Munchen (DE)

Correspondence Address: ROTHWELL, FIGG, ERNST & MANBECK, P.C., 1425 K STREET, N.W., SUITE 800, WASHINGTON, DC, 20005, US

(73) Assignee: PolyPat, Inc., San Diego, CA (US)

(21) Appl. No.: 11/653,352

(22) Filed: Jan. 16, 2007

Related U.S. Application Data
(63) Continuation of application No. 10/494,277, filed on Oct. 19, 2004, now abandoned.

(30) Foreign Application Priority Data
Oct. 30, 2001 (DE) 101 53 470.1
Oct. 8, 2002 (DE) 102 46 816.8

(51) Int. Cl. A61K 31/722; C12N 5/08; A61K 9/70

(52) U.S. Cl. 424/443; 514/55; 435/368

(57) ABSTRACT

The invention relates to the use of a biocompatible material based on chitosan and an acid, e.g. as flexible film or/and porous matrix, as remedy in the area of neurosurgery, in particular as nerve splint, and for repairing tendons and ligaments.

This application is a continuation of U.S. Ser. No. 10/494,277 which is a 35 USC § 371 National Phase Entry Application from PCT/EP2002/12112, filed Oct. 30, 2002.

The invention relates to the use of a biocompatible material based on chitosan and an acid, e.g. as flexible film or/and porous matrix, as remedy in the area of neurosurgery, in particular as nerve splint, and for repairing tendons and ligaments.

German patent application 199 48 120.2 discloses a method for producing a biocompatible three-dimensional matrix, wherein an aqueous solution of a chitosan and of an acid, which is present in excess, in particular a hydroxy carboxylic acid, is frozen, and the water is removed by sublimation under reduced pressure, with the excess acid being removed, in particular neutralized, before the freezing or after the removal of the water by sublimation. A matrix which can be obtained by the method and which can be used for producing implants is also disclosed.

German patent application 101 17 234.6 discloses biocompatible nonporous materials based on chitosan and an acid, in particular a hydroxy carboxylic. acid. These materials may for example be in the form of a film.

Based on this knowledge, it was the object of the present invention to provide novel applications for materials based on chitosan and an acid, in particular a hydroxy carboxylic acid.

A first aspect of the present invention therefore relates to the use of a biocompatible material based on chitosan and an acid, in particular a hydroxy carboxylic acid, as remedy in the area of neurosurgery, for example for extracorporeal or intracorporeal nerve reconstruction.

In a first embodiment, the material is in the form of a flexible, in particular nonporous, film. The film has a thickness of, preferably, 1 μm-200 μm, particularly preferably of 10 μm-50 μm and is obtainable by:
providing an aqueous solution of a chitosan and of an acid, in particular a hydroxy carboxylic acid, which is present in excess,
drying the solution without freezing and
removing excess acids before or/and after drying, preferably by neutralization.

The film can be produced in prefabricated strips with a width of, for example, 1 mm-10 mm. Alternatively, the required pieces of film can also be made as required, e.g. during an operation.

In a particularly preferred embodiment there is use of a film with a memory effect, i.e. with a preferred direction of curling. This means that the film tends, under the conditions of use, to curl or form rolls. The film with memory effect can be produced in a simple manner by removing the acid, e.g. neutralizing, from one side of the film article.

The film, in particular the film with memory effect, is suitable as nerve splint for wrapping round nerves. It has been found that, in particular, Schwann cells are able to grow well on the film. The nerve splint can also be employed for example in traumatology for initial management of transected nerve ends and in reconstructive surgery.

A further area of use of the film is for wrapping round tendons and ligaments, in which case it is beneficial to use a rolled film. It has been found that the union of severed tendons and ligaments is promoted by tension-free wrapping with a film of the invention. The enveloping can be adapted to the dimensions of the wrapped ligaments and tendons. Under these conditions, tenocytes, e.g. including human tenocytes, show a distinctly better proliferation than in other matrices.

The film can be used as carrier for a porous three-dimensional matrix. It is thus possible to provide biocompatible composite materials which include at least one biocompatible film as described above, and at least one biocompatible porous matrix. The biocompatible porous matrix is preferably based on chitosan and an acid, in particular a hydroxy carboxylic acid. However, it is also possible to use other porous biocompatible matrices.

In a further preferred embodiment, the biocompatible material based on chitosan and an acid is a porous matrix. It is particularly preferred to use a biocompatible porous matrix as disclosed in the German application 199 48 120.2, which is obtainable by:
providing an aqueous solution of a chitosan and of an acid, in particular a hydroxy carboxylic acid, which is present in excess,
freezing and drying the solution, in particular by sublimation under reduced pressure and
removing excess acid before or/and after the freezing, in particular by neutralization with a suitable base, e.g. NaOH.

The porous matrix can be employed for example as swab or/and tampon for medical or veterinary medical applications, e.g. as neurological swab, as matrix for uniting nerve ends and/or as tampon for abscess cavities. It has been found that the porous matrix is particularly suitable for the ingrowth of neurons. A further advantage of the porous matrix is the high swelling capacity to 10 times the initial weight or more.

The porous matrix can be produced in prefabricated pieces with a volume of, for example, 1-10 mm³. Alternatively, the pieces required can be made as required, e.g. during an operation.

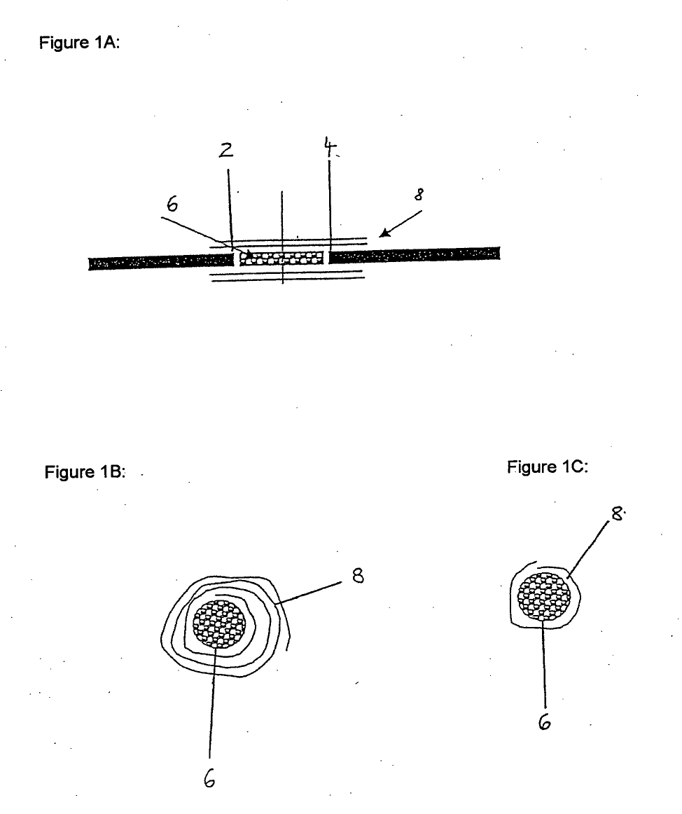

In a further embodiment, the material can be in the form of a combination of a flexible film and of a porous matrix. The film and the matrix can in this case be employed as separate components, for example for reconstructing severed nerves. An example of such a combination of materials, in which the film and matrix are employed as separate components, is shown in FIG. 1A, 1B and 1C. As shown in FIG. 1A, a porous matrix (6) is inserted between the ends (2, 4) of a severed nerve fiber. A film (8) is wound in one or more turns around the nerve filament. The film (8) can be fixed with a suitable adhesive, e.g. a fibrin glue or a tissue glue, for fixation to the nerve. FIG. 1B shows a cross section through the matrix (6) and the film (8), with the film being wound more than once. In FIG. 1C there is only one complete winding of the film (8) around the matrix (6).

Tendons and ligaments can be repaired in a manner analogous to that shown in FIG. 1. In addition, severed tendons and ligaments can also be anchored to bone. For this purpose, the bone, into which a depression can be cut where appropriate, is covered by a porous matrix into which tenocytes of the porous tendon or of the ligament can grow, where appropriate after wrapping in a rolled film, bringing about stable anchoring to the bone.

Yet a further preferred application of the film relates to a use as biohybrid implant, e.g. as capsular or tubular structure where appropriate in combination with the porous matrix for encapsulating cells, especially cells which can be electrically stimulated. In one embodiment, the implant is a neuron microprobe. In this case there is provision of an envelope consisting of the film, e.g. in the form of a bag or of a tubular structure, into which neuronal cells, which have been genetically manipulated where appropriate, are introduced. The envelope is implanted in the body and may serve, where appropriate after electrical stimulation to regenerate nerves, e.g. peripheral nerves, as pain pump (Erb et al., Exp. Neurol. 124, (1993), 372-376).

Moreover, the film can also serve as pain pump, where appropriate in combination with the matrix. This pain pump is an implant which has through external stimuli a controllable release of endorphins/enkephalins in the brain/subarachnoid space for the purpose of treating very severe chronic states of pain, as is the case for example in spinal disorders and tumor diseases.

In order to achieve genetically modified cells and the electrical stimulation, the film is used to sew a type of bag in which the biohybrid implant is located. Besides the film as external sheath, the matrix can additionally serve as carrier for the chromaffin cells which later release analgesic peptides in response to the electrical stimuli.

The chitosan-based material, especially the film, can additionally serve as covering for tissues and organs, e.g. the brain after injuries and/or during surgical procedures.

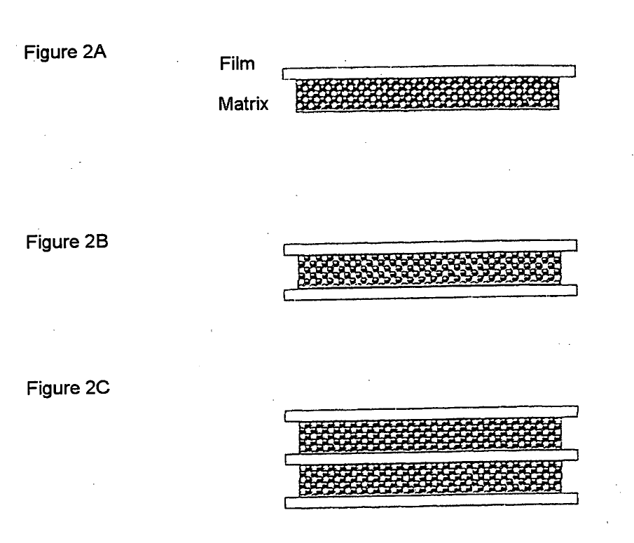

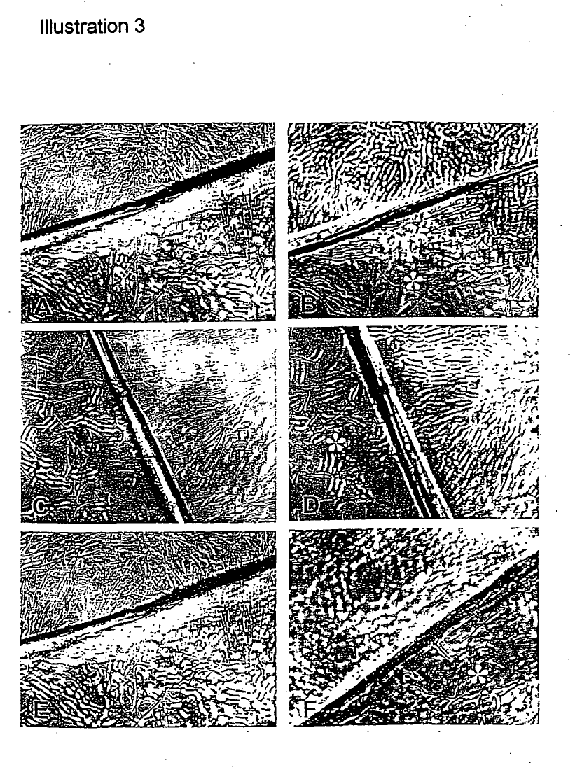

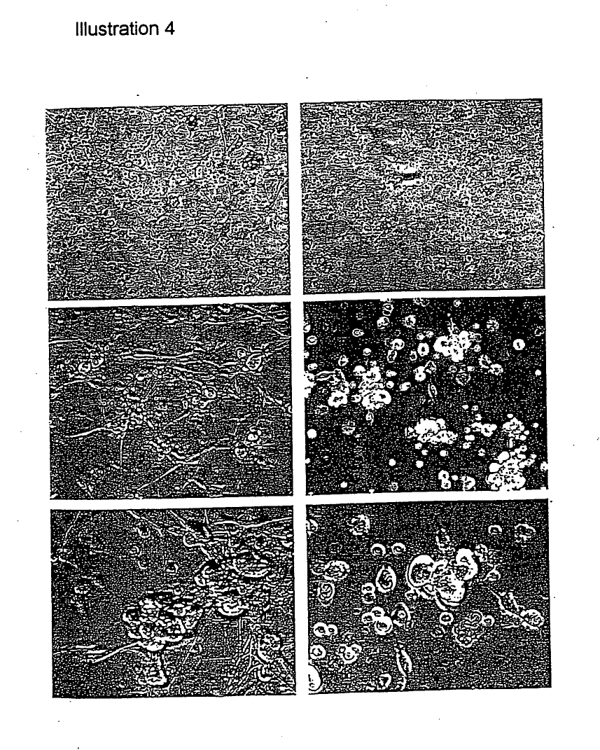

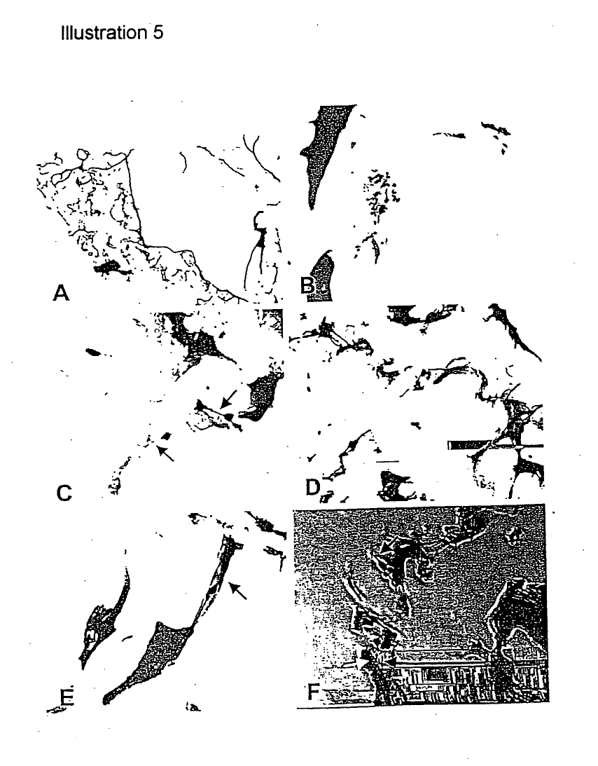

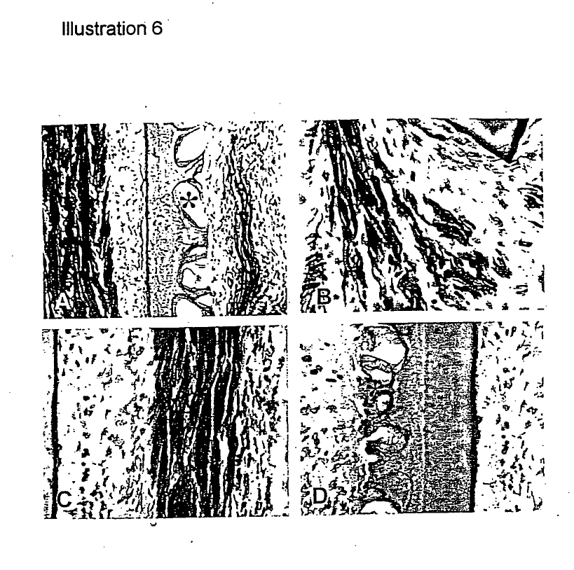

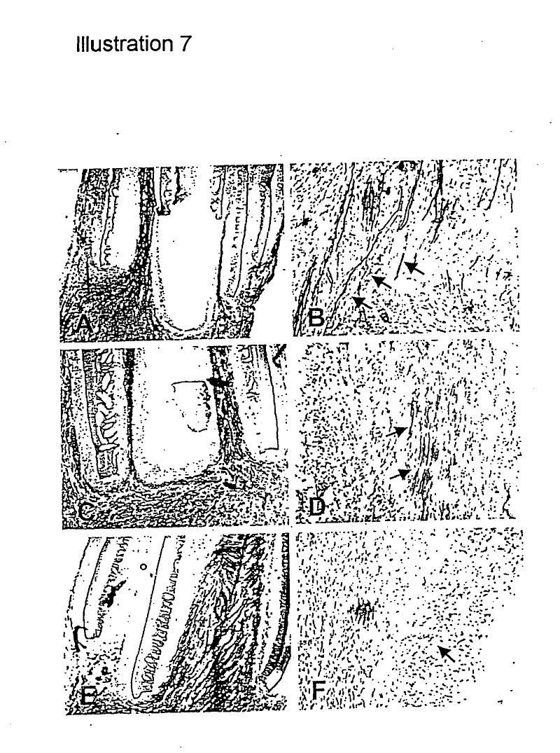

In an alternative embodiment it is also possible for film and matrix to be employed as composite components, with film and matrix each being disposed alternately in layers. Examples of such multilayer systems are depicted in FIG. 2A, 2B and 2C. An alternative possibility is also to dispose a nonporous film between two porous matrices.

The nonporous film of the invention, the porous matrix or the composite system based thereon can also be used for the in vitro cultivation of neuronal cells. In this case, the materials may comprise additional factors for cell growth, e.g. cytokines.

The porous matrix may where appropriate have anisotropic structures, for example fibers or/and chambers in parallel alignment. The anisotropic matrix is obtainable by:
providing an aqueous solution of a chitosan and of an acid, in particular a hydroxy carboxylic acid, which is present in excess,
anisotropic freezing and drying of the solution, in particular by sublimation under reduced pressure and
removing excess acid before or/and after the freezing.

The anisotropic freezing preferably comprises a freezing with use of structured cooling elements, e.g. tubes in direct or indirect contact with the matrix during the freezing process. The cooling elements may be elongate in order, for example, to obtain fibers or chambers in parallel alignment in the matrix. However, curved structures, e.g. simulations of the organ to be shaped, can also be used as cooling elements.

The anisotropic porous matrix can be employed in a biocompatible composite material system together with another material, for example with a biocompatible nonporous film. The anisotropic matrix, or the composite material system based thereon, can be employed for the in vitro cultivation of cells or as implant without previous cell colonization, corresponding to the applications mentioned above.

The matrices and films of the invention based on chitosan and acids are produced substantially by the method indicated in the German applications 199 48 120.2 and 101 17 234.6, unless indicated otherwise. Preferably, firstly an aqueous solution of a partially deacetylated chitosan and of an acid, which is present in excess, is prepared. Excess means in this connection that the aqueous solution has an acidic pH, preferably below pH ≦4. The free amino groups of the chitosan are at least in part protonated thereby, thus increasing the solubility in water. The amount of acid is not critical. It must merely be chosen so that the chitosan dissolves. Excessive addition of acid is avoided where possible, because excess acid must be removed again, and the working up is made difficult thereby when the amounts of acid are large. Favorable amounts of acid are those yielding a 0.05 to 1 N, preferably 0.1 to 0.5 N, in particular 0.1 to 0.3 N, solution. The amount of chitosan is preferably chosen to result in a 0.01 to 0.5 M, preferably 0.1 to 0.3 M, solution. The structure of the matrix, in particular the pore size thereof, can be influenced by the concentration of the chitosan solution. It is possible in this way to adapt the pore size of the matrix to the particular cell type with which the matrix is to be colonized.

Because chitosan is produced from natural sources it has no uniform molecular weight. The molecular weight may be between 20 kDa to more than 1000 kDa, depending on the source and method of processing.

The chitosan for producing the three-dimensional matrix is not subject to any restrictions in relation to its molecular weight. The aqueous chitosan solution is prepared by using an acid which is an inorganic acid or, preferably, an organic acid, particularly preferably an alkyl or aryl hydroxy carboxylic acid. Hydroxy carboxylic acids having 2 to 12 carbon atoms are particularly suitable, it being possible for one or more hydroxyl groups and one or more carboxyl groups to be present in the molecule. Specific examples are glycolic acid, lactic acid, malic acid, tartaric acid, citric acid and mandelic acid. Lactic acid is particularly preferred.

In producing a porous matrix, the solution of chitosan and acid is initially at least partially neutralized by adding base and then frozen or directly frozen without previous neutralization. Neutralization before freezing is preferred. The pH after the neutralization is generally 5.0 to 7.5, preferably 5.5 to 7.0 and in particular 6.0 to 7.0.

After the freezing, the water is removed by sublimation under reduced pressure, for example in the pressure range from 0.001 to 3 hPa.

To produce a nonporous film, the solution is not subjected to freezing and sublimation, but is dried without freezing at optionally elevated temperature or/and reduced pressure, and is preferably neutralized after drying. The resulting nonporous matrix has a high load-bearing capacity and extensibility in the moist state.

The large number of amino and hydroxyl groups makes the material modifiable as desired. In a preferred embodiment, ligands are covalently or noncovalently bound to the chitosan, preferably to the free amino groups of the chitosan. Ligands which can be used are, for example, growth factors, proteins, hormones, heparin, heparan sulfates, chondroit sulfates, dextran sulfates or a mixture of these substances. The ligands preferably serve to control and improve cell proliferation.

Cell growth on the matrix or the film is further improved if the matrix is coated with autologous fibrin.

The three-dimensional matrix can be colonized both by human and by animal cells (for example from horse, dog or shark). Shark cells are particularly suitable because they induce a negligible immunological response in the recipient.

The materials as described above can be employed in the human medical and veterinary sectors. Further areas of application are the use as disposable article, for example as swab.

The materials are sterilized before use in the cell culture, in order to guarantee freedom from germs. The sterilization can take place by thermal treatment, e.g. by autoclaving, steam treatment etc. or/and by irradiation, e.g. gamma-ray treatment. The sterilization preferably takes place in a physiologically tolerated buffered solution, e.g. in PBS, in order to ensure thorough wetting of the matrix or the film with liquid and the absence of major air inclusions.

When the cells are cultured, the material is degraded within a period of about 5-8 weeks or longer. The degradation times can be adjusted via the degree of deacetylation of the chitosan and the concentration of the material.

The invention is further to be explained by the following figures and examples.

### EXPLANATION OF THE FIGURES

Illustration 1 shows in FIGS. 1A, 1B and 1C combinations of materials in which film and matrix are employed as separate combinations.

Illustration 2 shows in FIGS. 2A, 2B and 2C alternative embodiments.

Illustration 3: pictures A and B: Schwann cells (rat adult, 10% FCS), uncoated; pictures C and D: PORN/laminin, pictures E and F: PLL; plane of focus: culture dish (A, C, E); film* (B, D, E).

Illustration 4: rat spinal ganglionic neuron cultures (P1), PORN-laminin coated plastic (left); uncoated film (right), 24 h in culture.

Illustration 5: spinal ganglionic neurons (diss. P1, rat) under serum-free conditions (+NGF), 3 days of culturing. Some neurons become detached from the matrix during the histological workup. Nevertheless, differentiated neurons with axons are found in association with the matrix (see arrows in C, E, F).

Illustration 6: Axonal regeneration (rat, adult, sciatic nerve) 8 weeks after implantation of a “wound” film graft. The wound film is incorporated in the connective tissue. The nature of the incorporation (no cells in the. cavities) suggests that the “fraying” on the inside of the film (A*, D*) is not attributable to postoperative enzymatic synthesis. Regenerating axons in some cases grow as large fascicles into the individual lamellae (proximal junction, see B).

Illustration 7: axional regeneration (rat, adult, sciatic nerve) of three experimental animals (A/B; C/D; E/F) 8 weeks after implantation of a “found” film graft. Survey magnifications (4×, A, C, E) of the proximal nerve-film junction, and detailed magnifications of the distal nerve stump (10×) with regenerated axons (see arrow).

### EXAMPLE 1

### Production of a Nonporous Film

A mixture of chitosan and lactic acid is prepared by the method described in Example 3 of DE 199 48 120.2. The solution is poured into a Petri dish, dried at 50° C. and, after a glass-clear film has formed, neutralized with 1 M sodium hydroxide solution to a pH of 7. The resulting film has a high load-bearing capacity and extensibility in the moist state.

A film with memory effect can be generated by specific addition of the sodium hydroxide solution onto one side.

### EXAMPLE 2

### In Vitro Cultivation of Schwann Cells and Neurons

Schwann cells and spinal ganglionic neurons were put onto the film or the matrix and cultured in vitro. The film is particularly suitable for culturing Schwann cells thereon (Illustration 3 and 4).

Neurons are successfully cultured in the matrix especially when the pore diameters are about 10-20 μm (Illustration 5).

### EXAMPLE 3

### Atraumatic Nerve Approximation Using a Film with Memory Effect

Principle and Surgical Method:

Two nerve stumps are connected by means of the self-curling chitosan film, and the ends are fixed using commercially available fibrin glue. The film is spread using forceps, and the nerve stumps are placed thereon. After removal of the forceps, the film curls up of its own accord (memory effect) and encloses the nerve ends.

Advantages of the Method:

No microsurgical suture is necessary, i.e. the procedure can be performed easily even by a clinician with no microsurgical experience, i.e. even by a traumatologist.

The curling up of the film avoids a pressure on the nerve ends: in the event of swelling, which regularly occurs after severance of a nerve, of the nerve stumps, the film is easily able to adapt to the increased diameter without exerting a pressure effect on the nerve ends. This avoids a substantial disadvantage of artificial nerve grafts customary at present, namely the secondary nerve damage from circular structures of constant diameter.

Results:

A total of 10 nerves was investigated after implantation in the rat model for eight weeks (Illustrations 6 and 7).

Results:

1. Ingrowth of regenerating nerve fibers into the grafts took place in all the animals investigated. The fibers grew between the lamellae of the curled up film.
2. The width of the grafts led to multiple curling. A single curling up would be ideal, so that the end result is a tube with only one slit.
3. The distal nerve stump was reached in all the animals investigated.
   Conclusions from the In Vivo Experiments:

A nerve splinting using a chitosan film with memory effect is possible. The nerve splinting allows nerve approximation even if a dehiscence is present between the nerve ends. At present, this still requires implantation of a nerve graft, e.g. from a cutaneous nerve of the leg. Coating with film with Schwann cells can achieve an increased rate of regeneration.

1. A method of treating neurosurgical injury comprising the step of applying a biocompatible material comprising chitosan and an acid over the injured neuronal organ.

2. The method of claim 1, wherein the method reconstructs extracorporeal or intracorporeal nerve.

3. The method of claim 1, wherein said material is in the form of a flexible, in particular nonporous, film.

4. The method of claim 3, wherein said film has a preferred direction of curling.

5. The method of claim 3, wherein said film is applied as nerve splint for wrapping round nerves.

6. The method of claim 3, wherein the method is growing Schwann cells on said film.

7. The method of claim 1, wherein said material is in the form of a porous matrix.

8. The method of claim 7, wherein said material is applied as neurological swab.

9. The method of claim 7, wherein said material is applied as matrix for uniting nerve ends.

10. The method of claim 9, wherein neurons grow into the porous matrix.

11. The method of claim 1, wherein said material is in the form of a combination of a flexible film and of a porous matrix.

12. The method of claim 11, wherein said film and matrix are employed as separate components.

13. The method of claim 11, wherein film and matrix are employed as composite component.

14. The method of claim 1, wherein said material is used as a neuron microprobe.

15. The use as claimed in claim 1 for producing a neuron microprobe.

16. A biocompatible material comprising chitosan and an acid in the form of a swab or tampon for abscess cavities.

17. A biocompatible material based on chitosan and an acid, comprising a film and a matrix, wherein said film and matrix form a composite component, and said film has a preferred direction of curling.

18. The biocompatible material of claim 17, wherein said film has a thickness in the range from 1 μm to 100 μm.
